(19) United States
(12) Patent Application Publication
(10) Pub. No.: US 2023/0416725 A1
(43) Pub. Date: Dec. 28, 2023

(54) SYSTEMS FOR GENE EDITING AND METHODS OF USE THEREOF

(71) Applicant: Rutgers, The State University of New Jersey, New Brunswick, NJ (US)

(72) Inventors: Peter J. Romanienko, Woodbridge, NJ (US); Ghassan Yehia, Bridgewater, NJ (US)

(73) Assignee: Rutgers, The State University of New Jersey, New Brunswick, NJ (US)

(21) Appl. No.: 18/245,256

(22) PCT Filed: Sep. 14, 2021
(86) PCT No.: PCT/US21/50222

§ 371 Date: Mar. 14, 2023

Related U.S. Application Data
(60) Provisional application No. 63/078,449, filed on Sep. 15, 2020.

(51) Int. Cl. C12N 15/10; C12N 9/22; C12N 15/113; C12N 5/0775

(52) U.S. Cl. CPC C12N 15/102; C12N 9/22; C12N 15/113; C12N 5/0662; C12N 2310/20

(57) ABSTRACT

This disclosure provides a novel gene editing method and system, termed dasCRISPR. dasCRISPR refines current approaches in gene editing by allowing an intended modification to be made in one genomic target while preventing modification to a second genomic target through protective sequestration. This balancing of repair and protection contributes to cellular survival by retention of one functional copy of a gene. Thus, dasCRISPR method as disclosed allows successful CRISPR gene editing of an intended modification at a target sequence by an active Cas polypeptide, while preserving specific genomic regions, protected by dCas polypeptide, from unintended modification

### CROSS-REFERENCE TO RELATED APPLICATIONS

This application claims priority under 35 U.S.C. § 119(e) to U.S. Provisional Patent Application No. 63/078,449, filed Sep. 15, 2020. The foregoing application is incorporated by reference herein in its entirety.

### FIELD OF THE INVENTION

This disclosure relates generally to systems and methods for gene editing using both an active Cas protein and a nuclease-deficient Cas (dCas) protein.

### BACKGROUND OF THE INVENTION

The discovery and use of clustered, regularly interspaced, short palindromic repeats (CRISPR)-associated endonuclease, e.g., Cas9, marks a significant breakthrough in the field of genetic engineering, with its ability to target and cut DNA at specific genomic loci in mammalian cells using a single guide RNA (gRNA). As a result, it has been successfully applied to various genome editing applications, including the genetic knock-out, knock-in, and correction of mutated genes, all of which can have significant therapeutic implications (Cong, L. et al. Science 339, 819-823 (2013)). The CRISPR/Cas system has also been adapted for sequence-specific control of gene expression, e.g., inhibition or activation of gene expression. Using particular Cas9 polypeptide variants that lack endonuclease activity, target genes can be repressed or activated (Qi et al., Cell, 2013, 152(5): 1173-7783, Perez-Pinera et al., Nat Methods, 2013, 10(10):973-976, Maeder et al., Nat Methods, 2013, 10(10):977-979, Gilbert et al., Cell, 2014, 159:647-661, O'Connell et al., Nature, 2014, 516:263-266).

CRISPR technology is a very efficient tool used in editing mouse genome to generate genetically engineered mouse models harboring a variety of mutations that could disrupt gene expression or lead to mutated proteins.

However, the CRISPR/Cas system has limitations. For example, a substantial number (about 25%) of mouse genes are essential for embryonic development, and another 7% are necessary for fertility and proper reproduction. Disruption of these genes may result in a reduced number of offspring or none at all with the desired outcome.

Given the above challenges, there is a pressing need for more effective gene editing systems.

### SUMMARY OF THE INVENTION

This disclosure addresses the need mentioned above in a number of aspects. In one aspect, this disclosure provides a system for gene editing, comprising: (i) a CRISPR-associated protein (Cas) polypeptide or a first Cas nucleotide sequence encoding a Cas polypeptide; (ii) a nuclease-deficient Cas (dCas) polypeptide or a second Cas nucleotide sequence encoding a dCas polypeptide; and (iii) a guide nucleotide sequence (e.g., guide RNA sequence or gRNA sequence) encoding or comprising a crRNA sequence capable of hybridizing with a first target sequence on a first allele and a second target sequence (which could be the same sequence as the first target) on a second allele (or subsequent duplicated alleles) and forming a complex with the Cas polypeptide and the dCas polypeptide. The Cas polypeptide/gRNA complex binds to the first target sequence on the first allele and induces a genetic modification in the first target sequence, whereas the dCas polypeptide/gRNA complex binds to the second target sequence on the second allele (or subsequent duplicated alleles) and protects the second target sequence from the genetic modification. In some embodiments, the genetic modification comprises an insertion of a stop codon to the first target sequence or a deletion that creates a downstream encoded stop codon through a sequence frameshift in the DNA.

In some embodiments, the first target sequence comprises one or more mutations. In some embodiments, the first target sequence and the second target sequence are identical, except that the first target sequence comprises one or more mutations. In some embodiments, the first target sequence and the second target sequence are identical or have slightly overlapping sequences.

In some embodiments, the guide nucleotide sequence together with the Cas polypeptide or the dCas polypeptide are delivered to a cell or an embryo as a ribonucleoprotein complex. In some embodiments, the Cas polypeptide and the dCas polypeptide, or the expression levels of the Cas polypeptide and the dCas polypeptide, have a ratio of between about 1:100 and about 100:1 (e.g., 1:80, 1:50, 1:10, 1:9, 1:8, 1:7, 1:6, 1:5, 1:4, 1:3, 1:2, 1:1, 10:1, 20:1, 50:1, 80:1). There are essentially unlimited ratios of Cas polypeptide to the dCas polypeptide. In some embodiments, the Cas polypeptide and the dCas polypeptide have a ratio of between about 1:10 and about 10:1. In some embodiments, the Cas polypeptide and the dCas polypeptide have a ratio of about 2:1, about 1:2, about 1:4, about 1:6, or about 1:8.

In some embodiments, the first Cas nucleotide sequence and the second Cas nucleotide sequence are located on the same vector. In some embodiments, the guide nucleotide sequence is located on the same vector with the first Cas nucleotide sequence or with the second Cas nucleotide. In some embodiments, the system further comprises a second guide nucleotide sequence, wherein the second guide nucleotide sequence and the second Cas nucleotide sequence are located on the same vector, and wherein the guide nucleotide sequence and the first Cas nucleotide sequence are located on the same vector.

In some embodiments, the first Cas nucleotide or the Cas polypeptide sequence and the second Cas nucleotide or the dCas polypeptide sequence are otherwise identical, except that the second Cas nucleotide or the dCas polypeptide comprises one or more mutations causing a deficiency in nuclease activity of the dCas polypeptide.

In some embodiments, the Cas polypeptide is selected from the group consisting of a Cas9 nuclease, a Cpf1 nuclease, a Cas12a nuclease, a Cas12e nuclease, a CasX nuclease, a Cas12d nuclease, a CasY nuclease, a Cas12b nuclease, a C2C1 nuclease, a Cas12c nuclease, a C2C3 nuclease, a C2C4 nuclease, a C2C5 nuclease, a C2C6 nuclease, a C2C7 nuclease, a C2C8 nuclease, a C2C9 nuclease, a C2C10 nuclease, a Cas13a nuclease, a Cas13b nuclease, and a Cas13c nuclease.

In some embodiments, the Cas polypeptide is a Cas9 nuclease. In some embodiments, the Cas9 nuclease is selected from the group consisting of Streptococcus pyogenes Cas9 (SpCas9), Staphylococcus aureus Cas9 (SaCas9), Neisseria meningitidis Cas9 (NmCas9), Actinomyces naeslundii Cas9 (AnCas9), and Streptococcus thermophilus Cas9 (StCas9).

In another aspect, this disclosure also provides a host cell or cell line or progeny thereof, an animal, or animal model comprising the system described above. In some embodiments, the host cell or cell line or progeny thereof comprises a stem cell or stem cell line.

In another aspect, this disclosure further provides a composition comprising the system or the host cell or cell line or progeny thereof, as described above.

In yet another aspect, this disclosure additionally provides a method of modifying a target sequence of interest. The method comprises delivering the system or the composition described above to the target sequence or a cell containing the target sequence and thereby inducing a modification in the target sequence. In some embodiments, the target sequence is located at genomic loci of interest.

In some embodiments, the target sequence is part of a gene, and the modification in the target sequence modulates the expression level and/or function of the gene. In some embodiments, the modification in the target sequence reduces the expression level and/or function of the gene.

In some embodiments, the gene is selected from the group consisting of p53, LOXL1, NOX4, SNX27, and Cathepsin B. In some embodiments, the modification on only one allele, while protecting the second allele, causes reduced DNA damage response (DDR) in hematopoietic stem cells and other cells, reduced cytokine expression, or reduced inflammation.

In some embodiments, the cell is a eukaryotic cell. In some embodiments, the cell is a plant, animal, or human cell. In some embodiments, the method comprises delivering the system via particles, vesicles, or one or more viral vectors. In some embodiments, the one or more viral vectors comprise an adenovirus-based vector, a lentivirus-based vector, or an adeno-associated virus-based vector.

In some embodiments, the target sequence comprises a genetic defect that is associated with a disease. In some embodiments, the disease is cancer, a genetic disease or a neurodegenerative disease.

In another aspect, this disclosure further provides a method of treating a disease of a subject caused by a genetic defect in a target sequence. The method comprises administering the system or the composition described above to a cell containing the target sequence in a subject in need thereof and thereby inducing a modification in the target sequence. In some embodiments, the target sequence is located at genomic loci of interest. In some embodiments, the target sequence is part of a gene, and the modification in the target sequence modulates the expression level of the gene. In some embodiments, the modification in the target sequence reduces the expression level of the gene. In some embodiments, the modification in the target sequence results in the expression of a modified amino acid sequence of an endogenous protein.

In yet another aspect, this disclosure also provides a method for blocking a nucleotide sequence from a modification (e.g., cleavage). The method comprises: contacting a nucleic acid molecule comprising a target sequence that is subject to protection from cleavage by an endonuclease with (i) a dCas polypeptide and (ii) a guide nucleotide sequence encoding or comprising a crRNA sequence capable of hybridizing with a target sequence, wherein a complex of the dCas polypeptide and the guide nucleotide sequence binds to the target sequence, thereby blocking the target sequence from being cleaved by the endonuclease.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A and 1B are schematic representations of dasCRISPR mode of action (deadCas Allelic Sequestration CRISPR). FIG. 1A shows that dasCRISPR provides a means to selectively block one allele from modification, by utilizing a dead Cas9 protein to bind and block a sequence allowing a functional Cas9 to target the remaining allele to induce a genetic change (left panel). If both alleles are inactivated (right panel) in the presence of only functional Cas9, deleterious mutations can cause lethality. FIG. 1B shows that dasCRISPR is used to reduce off-target activity (e.g., unwanted modifications) by Cas9 and thus increase editing efficiency.

FIG. 2 shows that dasCRISPR effectively increases the frequency of live births when targeting the mouse Acvrl1 locus. Experiment 1—summary of several attempts to generate live Acvrl1 R478X founders using functional Cas9 by electroporating 1 cell embryos (zygotes). Experiment 2—summary of attempts to generate live Acvrl1 R478X founders using functional Cas9 by microinjecting 1 cell of 2-cell embryos. Experiment 3—first attempt using dasCRISPR (dAS-CRISPR 1) to generate live Acvrl1 R478X founders by electroporating 1 cell embryos. Experiment 4—second attempt using dASCRISPR (dAS-CRISPR 2) to generate live Acvrl1 R478X founders by electroporating 1 cell embryos.

FIG. 3 shows that DasCrispr can rescue embryos from early embryonic lethality caused by active Cas9. A normal mouse embryo at E10.5 (right) compared to an Acvrl1 null E10.5 embryo, which has a smaller size and enlarged heart bud (arrow) were generated by co-electroporation of active Cas9 and dead Cas9 into mouse zygotes as conducted in Experiment 3 and 4 (FIG. 2)

### DETAILED DESCRIPTION OF THE INVENTION

This disclosure provides a novel method and system, termed dasCRISPR, for gene editing. The method utilizes both a nuclease-deficient (or catalytically inactivated) Cas protein (e.g., Cas9, Cas12) or related Cas-like protein and an active Cas protein or other endonuclease. The nuclease-deficient Cas protein blocks one DNA target site within a cell or embryo, while the active Cas protein (or other endonuclease) cuts the available, unoccupied site, thus allowing genetic modification of a single target allele. This method preserves one intact wild type allele, which allows the cell or embryo to survive. Thus, the method as disclosed allows successful completion of CRISPR gene editing that is hindered by generating mutations deleterious to animal/cell survival.

### A. dasCRISPR Gene Editing Systems

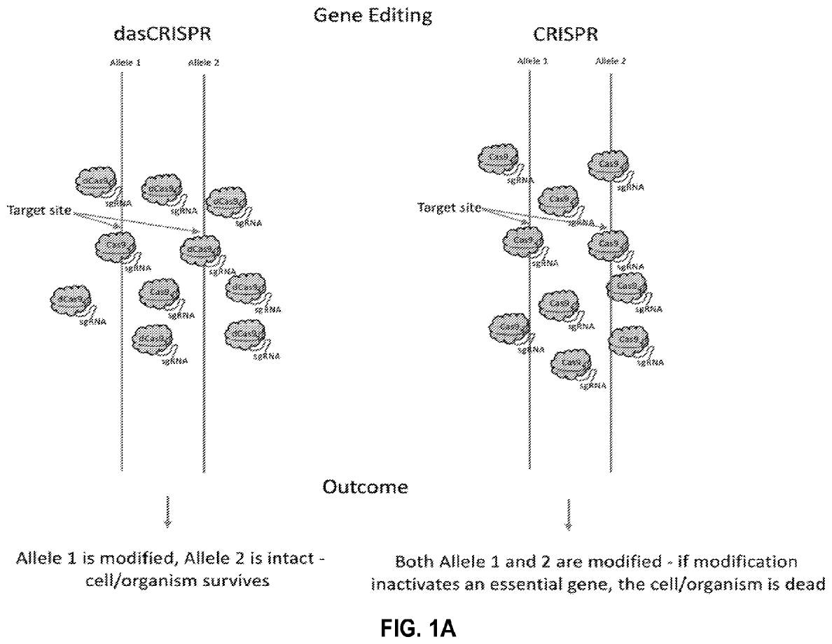

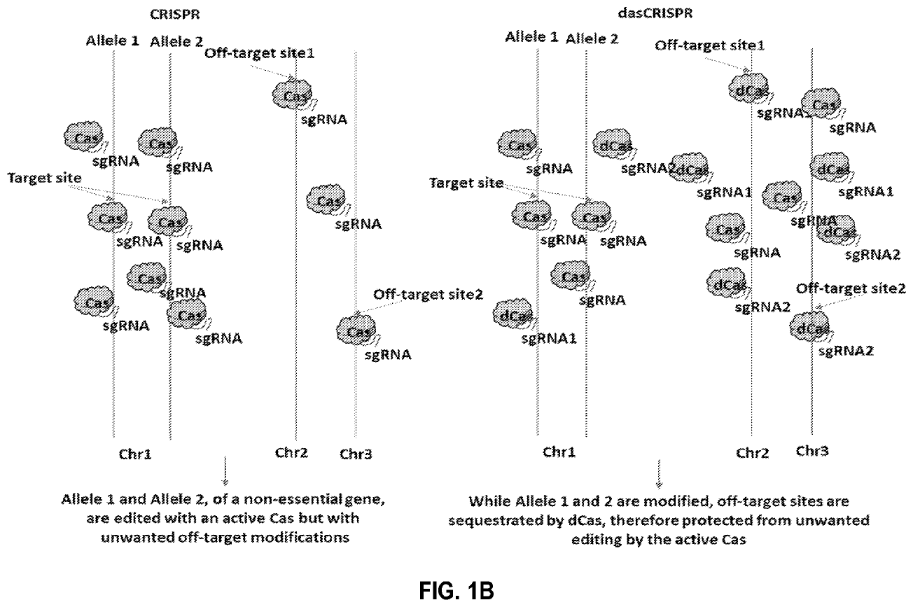

FIGS. 1A and 1B are schematic representations of dasCRISPR mode of action and its exemplary applications. As shown in FIG. 1A, dasCRISPR provides a means to selectively block one allele from modification, by utilizing a dead Cas9 (also called nuclease-deficient Cas9 or dCas9) protein to bind and block a sequence allowing a functional Cas9 to target the remaining allele to induce a genetic change (left panel). In comparison, in a typical CRISPR in which only functional Cas9 is present, both alleles will be inactivated and deleterious mutations can cause lethality (right panel).

FIG. 1B shows another application of dasCRISPR in which dasCRISPR is used to reduce off-target activity (e.g., unwanted modifications) by Cas9 and thus increase editing specificity. In particular, dCas is used to sequester off-target sites, thus blocking the functional Cas protein from modifying the off-target sites.

Accordingly, in one aspect, this disclosure provides a system for gene editing, comprising: (i) a Cas polypeptide or a first Cas nucleotide sequence encoding a Cas polypeptide or a variant/fragment thereof; (ii) a nuclease-deficient Cas (dCas) polypeptide or a second Cas nucleotide sequence encoding a dCas polypeptide or a variant/fragment thereof, and (iii) a guide nucleotide sequence (e.g., guide RNA sequence or gRNA sequence) encoding or comprising a crRNA sequence capable of hybridizing with a first target sequence on a first allele and a second target sequence on a second allele and forming a complex with the Cas polypeptide and the dCas polypeptide. The Cas polypeptide binds to the first target sequence on the first allele and induces a genetic modification in the first target sequence, whereas the dCas polypeptide binds to the second target sequence on the second allele and protects the second target sequence from the genetic modification. In some embodiments, the genetic modification comprises an insertion of a stop codon to the first target sequence.

In some embodiments, the first target sequence and the second target sequence are identical or have slightly overlapping sequences. In some embodiments, the first target sequence comprises one or more mutations. In some embodiments, the first target sequence and the second target sequence are otherwise identical, except that the first target sequence comprises one or more mutations.

In some embodiments, the active Cas polypeptide and the dCas polypeptide belong to different CRISPR CAS families and the corresponding gRNA sequences capable of hybridizing with a first target sequence on a first allele and a second target sequence on a second allele. For the active Cas polypeptide can be a Cas12 protein, whereas the dCas polypeptide can be a nuclease-deficient Cas9 polypeptide.

In some embodiments, the guide nucleotide sequence together with the Cas polypeptide or the dCas polypeptide are delivered to a cell or an embryo as a ribonucleoprotein complex. In some embodiments, knocking out or editing genes (e.g., essential genes) having one or more mutations can be achieved by adjusting the ratio of active Cas:dCas (e.g., Cas9:dCas9). In some embodiments, the Cas polypeptide and the dCas polypeptide, or the expression levels of the Cas polypeptide and the dCas polypeptide, have a ratio of between about 1:100 and about 100:1 (e.g., 1:80, 1:50, 1:10, 1:9, 1:8, 1:7, 1:6, 1:5, 1:4, 1:3, 1:2, 1:1, 10:1, 20:1, 50:1, 80:1). In some embodiments, the Cas polypeptide and the dCas polypeptide have a ratio of between about 1:10 and about 10:1. In some embodiments, the Cas polypeptide and the dCas polypeptide have a ratio of about 2:1, about 1:2, about 1:4, about 1:6, or about 1:8.

As used herein, the term “CRISPR” refers to a technique of sequence-specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing may utilize the pathways of non-homologous end-joining (NHEJ) or homologous recombination to perform the edits. Gene regulation refers to increasing or decreasing the production of specific gene products such as protein or RNA.

The term “gRNA” or “guide RNA” as used herein refers to the guide RNA sequences used to target specific genomic DNA sequences for editing employing the CRISPR technique. Techniques for designing gRNAs and donor polynucleotides for target specificity are well known in the art. For example, Doench, J., et al. Nature biotechnology 2014; 32(12): 1262-7; Mohr, S. et al. (2016) FEBS Journal 283: 3232-38; and Graham, D., et al. Genome Biol. 2015; 16: 260. gRNA comprises or alternatively consists essentially of, or yet further consists of a fusion polynucleotide comprising CRISPR RNA (crRNA) and trans-activating CRISPR RNA (tracrRNA); or a polynucleotide comprising CRISPR RNA (crRNA) and trans-activating CRISPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

In yet another aspect, this disclosure also provides a method for blocking a nucleotide sequence from a modification (e.g., cleavage). The method comprises: contacting a nucleic acid molecule comprising a target sequence that is subject to protection from cleavage by an endonuclease (e.g., restriction enzyme) with (i) a dCas polypeptide and (ii) a guide nucleotide sequence encoding or comprising a crRNA sequence capable of hybridizing with a target sequence, wherein a complex of the dCas polypeptide and the guide nucleotide sequence binds to the target sequence, thereby blocking the target sequence from being cleaved by the endonuclease.

a. Cas and dCas Polypeptides

In some embodiments, the Cas polypeptide can be a variant/fragment of a Cas nuclease. Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, fragments thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref Seq. No. NP_269215 and the amino acid sequence of Streptococcus thermophilus wild-type Cas9 polypeptide is set forth, e.g., in NBCI Ref. Seq. No. WP_011681470. CRISPR-related endonucleases that are useful in the present invention are disclosed, e.g., in U.S. Application Publication Nos. 2014/0068797, 2014/0302563, and 2014/0356959.

In some embodiments, the Cas polypeptide is selected from the group consisting of a Cas9 nuclease, a Cpf1 nuclease, a Cas12a nuclease, a Cas12e nuclease, a CasX nuclease, a Cas12d nuclease, a CasY nuclease, a Cas12b nuclease, a C2C1 nuclease, a Cas12c nuclease, a C2C3 nuclease, a C2C4 nuclease, a C2C5 nuclease, a C2C6 nuclease, a C2C7 nuclease, a C2C8 nuclease, a C2C9 nuclease, a C2C10 nuclease, a Cas13a nuclease, a Cas13b nuclease, and a Cas13c nuclease.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Hyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides Capnoxytophaga ochracea, Rhodopseudomonas palustris, Prevotella micans, Prevotella ruminicola, Flavobacterium columnare, Aminomonas paucivorans, Rhodospirillum rubrum, Candidatus Pumceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihomins, Wolinella succinogenes, and Francisella novicida.

In some embodiments, the Cas polypeptide is a Cas9 nuclease. Cas9 is an RNA-guided double-stranded DNA-binding nuclease protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species. In some embodiments, the Cas9 nuclease is selected from the group consisting of Streptococcus pyogenes Cas9 (SpCas9), Staphylococcus aureus Cas9 (SaCas9), Neisseria meningitidis Cas9 (NmCas9), Actinomyces naeslundii Cas9 (AnCas9), and Streptococcus thermophilus Cas9 (StCas9).

In some embodiments, the Cas polypeptide or the dCas polypeptide comprises one or more nuclear localization signals (NLSs).

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, a fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made.

In some embodiments, a variant of a Cas protein (e.g., Cas9) may include one or more conservative modifications. The Cas protein variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The Cas protein with one or more conservative modifications may retain the desired functional properties, which can be tested using the functional assays known in the art.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used (See www.ncbi.nlm.nih.gov).

In some embodiments, a variant of a Cas protein (e.g., Cas9) can be conjugated or linked to a detectable tag or a detectable marker (e.g., a radionuclide, a fluorescent dye, or an MRI-detectable label). In some embodiments, the detectable tag can be an affinity tag. The term “affinity tag,” as used herein, relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of a variant of a Cas protein. The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

In some embodiments, the linker can be a peptide linker and a non-peptide linker. Examples of the peptide linker may include [Ser(Gly)n]m or [Ser(Gly)n]mSer, where n may be an integer between 1 and 20, and m may be an integer between 1 and 10. For example, the peptide linker can be SerGly, SerGlySer, SerGlyGly, SerGlyGlySer (SEQ ID NO: 49), SerGlyGlyGly, (SEQ ID NO: 50) SerGlyGlyGlySer (SEQ ID NO: 51), SerGlyGlyGlyGly (SEQ ID NO: 52), SerGlyGlyGlyGlySer (SEQ ID NO: 53), SerGlyGlyGlyGlyGly (SEQ ID NO: 54), SerGlyGlyGlyGlyGlySer (SEQ ID NO: 55), SerGlyGlyGlyGlyGlyGly (SEQ ID NO: 56), and SerGly GlySerGlyGlyGlyGlySer (SEQ ID NO: 57).

As used herein, the term “non-peptide linker” refers to a biocompatible polymer composed of two or more repeating units linked to each other, in which the repeating units are linked to each other by any non-peptide covalent bond. This non-peptidyl linker may have two ends or three ends. Examples of the non-peptidyl linker may include, without limitation, polyethylene glycol, polypropylene glycol, a copolymer of ethylene glycol with propylene glycol, polyoxyethylated polyol, polyvinyl alcohol, polysaccharide, dextran, polyvinyl ethyl ether, biodegradable polymers such as polylactic acid (PLA) and polylactic-glycolic acid (PLGA), lipid polymers, chitins, hyaluronic acid, aptamers and combinations thereof.

In some embodiments, a variant of a Cas protein (e.g., Cas9) can be fused to a fusion partner through crosslinking with a crosslinking agent, e.g., crosslinker. Crosslinkers are reagents having reactive ends to specific functional groups (e.g., primary amines or sulfhydryls) on proteins or other molecules. Crosslinkers are capable of joining two or more molecules by a covalent bond. Crosslinkers include but are not limited to amine-to-amine crosslinkers (e.g., disuccinimidyl suberate (DSS)), amine-to-sulfhydryl crosslinkers (e.g., N-γ-maleimidobutyryl-oxysuccinimide ester (GMBS)), carboxyl-to-amine crosslinkers (e.g., dicyclo-hexylcarbodiimide (DCC)), sulfhydryl-to-carbohydrate crosslinkers (e.g., N-β-maleimidopropionic acid hydrazide (BMPH)), sulfhydryl-to-sulfhydryl crosslinkers (e.g., 1,4-bismaleimidobutane (BMB)), photoreactive crosslinkers (e.g., N-5-azido-2-nitrobenzoyloxysuccinimide (ANB-NOS)), chemoselective ligation crosslinkers (e.g., NHS-PEG4-Azide).

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In some embodiments, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). In some embodiments, Cas9 and other Cas9 proteins may also have mutations that enhance specificity, i.e., high-fidelity Cas9, these can be used in conjunction as well with dCas type proteins.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In some embodiments, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, international patent publication No. WO 2013/176772. The dCas9 enzyme can contain a mutation at D10, E762, H983 or D986, as well as a mutation at H840 or N863. In some instances, the dCas9 enzyme contains a D10A or D10N mutation. Also, the dCas9 enzyme can include a H840A, H840Y, or H840N. In some embodiments, the dCas9 enzyme of the present invention comprises D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA in a site-specific manner. As a result, dCas9 can still be guided to a target polynucleotide sequence by a DNA-targeting RNA sequence of the subject polynucleotide (e.g., gRNA), as long as it retains the ability to interact with the Cas-binding sequence of the subject polynucleotide (e.g., gRNA).

In some embodiments, a nucleotide sequence encoding the Cas or dCas nuclease is present in a recombinant expression vector. In some embodiments, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Depending on the target cell/expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

In some embodiments, the first Cas nucleotide sequence and the second Cas nucleotide sequence are located on the same vector. In some embodiments, the guide nucleotide sequence is located on the same vector with the first Cas nucleotide sequence or with the second Cas nucleotide. In some embodiments, the system further comprises a second guide nucleotide sequence, wherein the second guide nucleotide sequence and the second Cas nucleotide sequence are located on the same vector, and wherein the guide nucleotide sequence and the first Cas nucleotide sequence are located on the same vector.

The Cas or dCas nuclease and variants or fragments thereof can be introduced into a cell (e.g., an in vitro cell such as a primary cell for ex vivo therapy, or an in vivo cell such as in a patient) as a Cas polypeptide or a variant or fragment thereof, an mRNA encoding a Cas polypeptide or a variant or fragment thereof, or a recombinant expression vector comprising a nucleotide sequence encoding a Cas polypeptide or a variant or fragment thereof.

b. Guide RNA (or gRNA)

A guide RNA may be composed of two RNAs, i.e., a CRISPR RNA (crRNA) and a trans-activating crRNA (tracrRNA). In some embodiments, the guide RNA may be a single-chain RNA (sgRNA) prepared by fusion of major parts of a crRNA and a tracrRNA. In some embodiments, the guide RNA is a single-chain RNA like in the case of Cas12a.

The nucleic acid sequence of the guide RNA can be any polynucleotide sequence having sufficient complementarity with a target polynucleotide sequence (e.g., target DNA sequence) to hybridize with the target sequence and direct sequence-specific binding of a CRISPR complex to the target sequence. In some embodiments, the degree of complementarity between a guide sequence of the guide RNA and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows-Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a guide sequence is about 20 nucleotides in length. In other instances, a guide sequence is about 15 nucleotides in length. In other instances, a guide sequence is about nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

In some embodiments, the guide RNA comprises a synthetic nucleic acid sequence (e.g., synthetic RNA molecule). In some embodiments, the guide RNA comprises one or more modifications.

The term “modification” in the context of an oligonucleotide or polynucleotide includes but is not limited to (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages. The term “modified nucleotide” generally refers to a nucleotide having a modification to the chemical structure of one or more of the base, the sugar, and the phosphodiester linkage or backbone portions, including nucleotide phosphates. The terms “Z” and “P” refer to the nucleotides, nucleobases, or nucleobase analogs are described, for example, in Yang, Z., et al., Nucleic Acids Res., 34, 6095-101 (2006), the disclosure of which is hereby incorporated by reference in its entirety.

In some embodiments, the one or more modifications may include 2′-O-methyl moiety, a Z base, a 2′-deoxynucleotide, a phosphorothioate internucleotide linkage, a phosphonoacetate (PACE) internucleotide linkage, a thiophosphonoacetate (thioPACE) internucleotide linkage, or combinations thereof. In some embodiments, the one or more modifications comprise one or more modifications selected from the group consisting of a 2′-O-methyl nucleotide with a 3′-phosphorothioate group, a 2′-O-methyl nucleotide with a 3′-phosphonoacetate group, a 2′-O-methyl nucleotide with a 3′-thiophosphonoacetate group, or a 2′-deoxynucleotide with a 3 ′-phosphonoacetate group. In some embodiments, the one or modifications comprises a 2-thiouracil (2-thioU), a 4-thiouracil (4-thioU), a 2-aminoadenine, a 2′-o-methyl, a 2′-fluoro, a 5-methyluridine, a 5-methylcytidine, or a locked nucleic acid modification (LNA).

### B. Cells, Compositions, and Kits

a. Cells

In another aspect, this disclosure also provides a host cell or cell line or progeny thereof comprising the system described above. In some embodiments, the host cell or cell line or progeny thereof comprises a stem cell or stem cell line.

The term “cell” as used herein may refer to either a prokaryotic or eukaryotic cell, optionally obtained from a subject or a commercially available source.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect, and mammalian cells. Non-limiting examples of eukaryotic cells or hosts include simian, bovine, porcine, murine, rat, avian, reptilian, and human, e.g., HEK293 cells and 293T cells.

“Prokaryotic cells” that usually lack a nucleus or any other membrane-bound organelles and are divided into two domains, bacteria, and archaea. In addition to chromosomal DNA, these cells can also contain genetic information in a circular loop called on episome. Bacterial cells are very small, roughly the size of an animal mitochondrion. Prokaryotic cells feature three major shapes: rod-shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

b. Pharmaceutical Compositions

The disclosed gene editing system can be incorporated into pharmaceutical compositions suitable for administration. The pharmaceutical compositions generally comprise the system described above and a pharmaceutically acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the nanoparticle construct, use thereof in the compositions is contemplated. Supplementary active compounds can also be incorporated into the compositions.

A pharmaceutical composition is formulated to be compatible with its intended route of administration. Solutions or suspensions used for intradermal or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride in the composition. Prolonged absorption of the injectable compositions can be brought about by including in the composition a compound which delays absorption, e.g., aluminum monostearate and gelatin.

c. Kits

This disclosure further provides kits containing one or more components (e.g., Cas, dCas, guide RNA) of the system described above. In some embodiments, the kit can include one or more other reaction components. In such a kit, an appropriate amount of one or more reaction components is provided in one or more containers or held on a substrate.

Examples of additional components of the kits include, but are not limited to, one or more host cells, one or more reagents for introducing foreign nucleotide sequences into host cells, one or more reagents (e.g., probes or PCR primers) for detecting expression of the RNA or protein or verifying the target nucleic acid's status, and buffers or culture media for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, RNAs, probes, and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits, it is preferred that they are provided and maintained below 0° C., preferably at or below −20° C., or otherwise in a frozen state.

A kit or system may contain, in an amount sufficient for at least one assay, any combination of the components described herein. In some applications, one or more reaction components may be provided in pre-measured single-use amounts in individual, typically disposable, tubes or equivalent containers. The amount of a component supplied in the kit can be any appropriate amount and may depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for the use of the components.

d. Methods and Uses

This disclosure also encompasses methods and uses of the gene editing systems described herein for modifying a target DNA sequence (e.g., a chromosomal sequence) or target RNA sequence, e.g., for altering or manipulating the expression of one or more genes or the one or more gene products, in prokaryotic or eukaryotic cells, in vitro, in vivo, or ex vivo. The disclosed gene editing system provides an effective means for modifying (e.g., deleting, inserting, translocating, inactivating, activating) a target DNA (double-stranded, linear or supercoiled) in a multiplicity of cell types. The system selectively modifies a single target allele, while preserving one intact wild type allele which allows the cell or embryo to survive. Thus, the disclosed gene editing systems have a broad spectrum of applications in, e.g., gene therapy, drug screening, disease diagnosis, and prognosis.

As used herein, “target,” “targets” or “targeting” refers to partial or no breakage of the covalent backbone of polynucleotide. In some embodiments, a deactivated Cas protein (or dCas) targets a nucleotide sequence after forming a DNA-bound complex with a guide RNA. Because the nuclease activity of the dCas is entirely or partially deactivated, the dCas binds to the sequence without cleaving or fully cleaving the sequence.

a. Methods of Modifying Expression of a Target Polynucleotide

In another aspect, this disclosure additionally provides a method of modifying a target sequence of interest. The method comprises delivering the system or the composition described above to the target sequence or a cell containing the target sequence and thereby inducing a modification in the target sequence. In some embodiments, the target sequence is located at genomic loci of interest. In some embodiments, the cell is a eukaryotic cell, such as a plant, animal, or human cell.

In some embodiments, the target sequence is part of a gene, and the modification in the target sequence modulates the expression level of the gene. In some embodiments, the modification in the target sequence reduces the expression level of the gene. In some embodiments, the modification in the target sequence results in the expression of a modified amino acid sequence of an endogenous protein.

In some embodiments, the gene is selected from the group consisting of p53, LOXL1, NOX4, SNX27, and Cathepsin B. In some embodiments, the modification on only one allele, while protecting the second allele, causes reduced DNA damage response (DDR) in hematopoietic stem cells and other cells, reduced cytokine expression, or reduced inflammation.

In some embodiments, the method of modifying a target polynucleotide comprises delivering the system or the composition, as described above, to a target sequence or a cell containing the target sequence. In some embodiments, following formation of a complex between the gRNA and the CRISPR-Cas protein and hybridization of the crRNA to one or more nucleic acid of the target sequence, the CRISPR-Cas protein induces a modification (e.g., cleavage) of the target sequence.

The target polynucleotide has no sequence limitation. In some embodiments, the target polynucleotide sequence is followed by or preceded a PAM sequence. In some embodiments, the target polynucleotide sequence does not contain a PAM sequence (or PAMless CRISPR. (Walton et al., Science 17 Apr. 2020: Vol. 368, Issue 6488, pp. 290-296). Other examples of PAM sequences are given above, and the skilled person will be able to identify further PAM sequences for use with a given CRISPR protein. The target polynucleotide can be in the coding region of a gene, in an intron of a gene, in a control region between genes, etc. The gene can be coding or non-coding.

The target polynucleotide can be any polynucleotide endogenous or exogenous to the cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide).

The method further comprises maintaining the cell or embryo under appropriate conditions such that the gRNA guides the Cas protein to the targeted site in the target sequence to modify the target sequence. In general, the cell can be maintained under conditions appropriate for cell growth and/or maintenance. Suitable cell culture conditions are well known in the art and are described, for example, in Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001), Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the proteins and RNA scaffold, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into a uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal-derived from the embryo. Such an animal would comprise the modified chromosomal sequence in every cell of the body or be mosaic with some portion of cells possessing the modified chromosomal sequence.

In some embodiments, the method comprises delivering the system via electroporation, particles, vesicles, or one or more viral vectors. In some embodiments, the one or more viral vectors comprise an adenovirus-based vector, a lentivirus-based vector, or an adeno-associated virus-based vector.

In some embodiments, the modification comprises cleaving one or two strands at the location of the target sequence by a Cas protein. In some embodiments, the modification results in decreased or increased transcription of a target gene. In some embodiments, the method further comprises repairing the cleaved target polynucleotide by homologous recombination with an exogenous template polynucleotide, wherein the repair results in a mutation comprising an insertion, deletion, or substitution of one or more nucleotides of the target polynucleotide. In some embodiments, the mutation results in one or more amino acid changes in a protein expressed from a gene comprising the target sequence. In some embodiments, the modification takes place in the eukaryotic cell in cell culture. In some embodiments, the method further comprises isolating the eukaryotic cell from a subject prior to the modification. In some embodiments, the method further comprises returning the eukaryotic cell and/or cells derived therefrom to the subject.

Methods for introducing polypeptides and nucleic acids into a target cell (host cell) are known in the art, and any known method can be used to introduce a nuclease or a nucleic acid (e.g., a nucleotide sequence encoding the nuclease, a DNA-targeting RNA (e.g., a modified single guide RNA), a donor repair template for homology-directed repair (HDR), etc.) into a cell, e.g., a primary cell such as a stem cell, a progenitor cell, or a differentiated cell. Non-limiting examples of suitable methods include electroporation, viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments, the components of CRISPR/Cas-mediated gene regulation can be introduced into a cell using a delivery system. In some embodiments, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct) and one or more nucleic acid components such as a DNA-targeting RNA (e.g., a modified single guide RNA) and/or a donor repair template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

b. Methods of Generating a Model Eukaryotic Cell

In one aspect, this disclosure provides a method of generating a model eukaryotic cell comprising a mutated disease gene, which can be any gene associated with an increase in the risk of having or developing a disease. In some embodiments, the method comprises introducing a system or a composition of the present invention into a eukaryotic cell.

In some embodiments, the cleavage comprises cleaving one or two strands at the location of the target sequence by a Cas protein (e.g., Cas nickase). In some embodiments, the cleavage results in decreased or increased transcription of a target gene. In some embodiments, the method further comprises repairing the cleaved target polynucleotide, for example, by Homology Directed Repair (HDR) mechanisms with an exogenous template polynucleotide, wherein the repair results in a mutation comprising an insertion, deletion, or substitution of one or more nucleotides of the target polynucleotide. In some embodiments, the mutation results in one or more amino acid changes in protein expression from a gene comprising the target sequence.

A variety of eukaryotic cells are suitable for use in the method. For example, the cell can be a human cell, a non-human mammalian cell, a non-mammalian vertebrate cell, an invertebrate cell, an insect cell, a plant cell, a yeast cell, or a single-cell eukaryotic organism. A variety of embryos are suitable for use in the method. For example, the embryo can be a 1-cell, 2-cell, or 4-cell human or non-human mammalian embryo. Exemplary mammalian embryos, including one-cell embryos, such as mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells, and others. In exemplary embodiments, the cell is a mammalian cell or the embryo is a mammalian embryo. In some embodiments, the non-human mammal cell may include, but not limited to, primate bovine, ovine, porcine, canine, rodent, Leporidae such as monkey, cow, sheep, pig, dog, rabbit, rat or mouse cell. In some embodiments, the cell may be a non-mammalian eukaryotic cell, such as poultry bird (e.g., chicken), vertebrate fish (e.g., salmon) or shellfish (e.g., oyster, clam, lobster, shrimp) cell. In some embodiments, the non-human eukaryote cell is a plant cell. The plant cell may be of a monocot or dicot or of a crop or grain plant such as cassava, corn, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

c. Methods of Developing a Biologically Active Agent

In another aspect, this disclosure provides a method for developing a biologically active agent that modulates a cell signaling event associated with a disease gene, which can be any gene associated with an increase in the risk of having or developing a disease. In some embodiments, the method comprises (a) contacting a test agent with a model cell, as described above; and (b) detecting a change in a readout that is indicative of a reduction or an augmentation of a cell signaling event associated with the mutation in the disease gene, thereby developing the biologically active agent that modulates the cell signaling event associated with the disease gene.

d. Methods of Treatment

In another aspect, this disclosure further provides a method of treating a disease of a subject caused by a genetic defect in a target sequence. The method comprises administering the system or the composition described above to a cell containing the target sequence in a subject in need thereof and thereby inducing a modification in the target sequence. In some embodiments, the target sequence is located at genomic loci of interest. In some embodiments, the target sequence is part of a gene, and the modification in the target sequence modulates the expression level of the gene. In some embodiments, the modification in the target sequence reduces the expression level of the gene.

The above-described systems or compositions can be used in a therapeutic method of treatment. The therapeutic method of treatment may comprise gene or genome editing, or gene therapy. In one aspect, this disclosure provides a method of treating a subject in need thereof, comprising inducing gene editing by delivering to a cell in the subject a system or a composition of the present invention. In some embodiments, the method comprises inducing transcriptional activation or repression by delivering to a cell in the subject a system or a composition of the present invention.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention.

In some embodiments, the method further comprises administering to the subject a recombinant donor repair template. In some embodiments, the recombinant donor repair template comprises two nucleotide sequences comprising two non-overlapping, homologous portions of the target gene, wherein the nucleotide sequences are located at the 5′ and 3′ ends of a nucleotide sequence corresponding to the target gene to undergo genome editing. In other instances, the recombinant donor repair template comprises a synthetic single-stranded oligodeoxynucleotide (ssODN) template comprising a nucleotide sequence encoding a mutation to correct a single nucleotide polymorphism (SNP) in the target gene, and two nucleotide sequences comprising two non-overlapping, homologous portions of the target gene, wherein the nucleotide sequences are located at the 5′ and 3′ ends of the nucleotide sequence encoding the mutation. The donor template may also be RNA (modified, synthetic) or expressed in the cell with the intention of that RNA being a donor template.

In some embodiments, the Cas polypeptide, the dCas polypeptide, the guide RNA, and/or the recombinant donor repair template are administered to the subject with a pharmaceutically acceptable carrier.

In some embodiments, the Cas polypeptide, the dCas polypeptide, the guide RNA, and/or the recombinant donor repair template are administered to the subject via a delivery system selected from the group consisting of a nanoparticle, a liposome, a micelle, a virosome, a nucleic acid complex, and a combination thereof. In some embodiments, the nucleic acid complex comprises the guide RNA complexed with the Cas polypeptide.

In some embodiments, the Cas polypeptide, the dCas polypeptide, the guide RNA, and/or the recombinant donor repair template are administered to the subject via a delivery route selected from the group consisting of oral, intravenous, intraperitoneal, intramuscular, intradermal, subcutaneous, intra-arteriole, intraventricular, intracranial, intralesional, intraocular, intrathecal, topical, transmucosal, intranasal, and a combination thereof.

Many devastating human diseases have one common cause: genetic alteration or mutation. The disease-causing mutations in patients are either acquired through inheritance from their parents or are caused by environmental factors. These diseases include, but are not limited to, the following categories. First, some genetic disorders are caused by germline mutations. One example is cystic fibrosis, which is caused by mutations at the CFTR gene inherited from parents. A second suppressor mutation in the mutant CFTR can partially restore the function of CFTR protein in somatic tissues. Other example genetic diseases caused by a point genetic mutation that can be corrected by the disclosed technology include Gaucher's disease, alpha trypsin deficiency disease, sickle cell anemia, to name a few. Second, some diseases, such as chronic viral infectious diseases, are caused by exogenous environmental factors and resulting in genetic alterations. One example is AIDS, which is caused by insertion of the human HIV viral genome into the genome of infected T-cells. Third, some neurodegenerative diseases involve genetic alterations. One example is Huntington's disease, which is caused by expansion of CAG tri-nucleotide in the huntingtin gene of affected patients. Finally, cancers are caused by various somatic mutations accumulated in cancer cells. Therefore, correcting the disease-causing genetic mutations, or functionally correcting the sequence, provides an appealing therapeutic opportunity to treat these diseases.

Somatic genetic editing is an appealing strategy for many human diseases. Through precise editing of the target DNA or RNA sequence, the CRISPR-Cas system can correct the mutated genes in genetic disorders, inactivate the viral genome in the infected cells, eliminate the expression of the disease-causing protein in neurodegenerative diseases, or silence the oncogenic protein in cancers. Accordingly, the system and method disclosed in this disclosure can be used in correcting underlying genetic alterations in diseases, including the above mentioned genetic disorders, chronic infectious diseases, neurodegenerative diseases, and cancer.

Genetic Diseases

It is estimated that over six thousand genetic diseases are caused by known genetic mutations. Correcting the underlying disease-causing mutations in the pathological tissues/organs can provide alleviation or cure to the diseases. For example, cystic fibrosis affects 1 out of every 3,000 people in the US. It is caused by inheritance of a mutated CFTR gene, and 70% of the patients have the same mutation, deletion of a tri-nucleotide leading to a deletion of phenylalanine at position 508 (called Δ Phe 508). Δ Phe 508 leads to the mislocation and degradation of CFTR. The system and method disclosed in this invention can be used to convert a Val 509 residue (GTT) to Phe 509 (TTT) in affected tissues (lung), thereby functionally correcting the Δ Phe 508 mutation. In addition, a second suppressor mutation (such as R553Q or R553M or V510D) in the mutant A Phe 508 CFTR can partially restore the function of CFTR protein in somatic tissues.

In some embodiments, the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation disease or disorders, inflammation, immune-related diseases or disorders, metabolic diseases, liver diseases and disorders, kidney diseases and disorders, muscular/skeletal diseases and disorders, neurological and neuronal diseases and disorders, cardiovascular diseases and disorders, pulmonary diseases and disorders, ocular diseases and disorders, and viral infections (e.g., HIV infection).

Chronic Infectious Diseases

The system and method, as disclosed, can also be used to specifically inactivate any gene in a viral genome that is incorporated into human cells/tissues. For example, the system and method disclosed in this invention allow one to create a stop codon for early termination of translation of the essential viral genes, and thereby remediate or cure the chronic debilitating infectious diseases. For example, current AIDS therapies can reduce viral load, but cannot totally eliminate dormant HIV from positive T cells. The system and method disclosed herein can be used to permanently inactivate one or two essential HIV gene expression in the integrated HIV genome in human T-cells by introducing one or two stop codons. Another example is the hepatitis B virus (HBV). The system and method disclosed here can be used to specifically inactivate one or two essential HBV genes, which are incorporated into the human genome, and silence HBV life-cycle.

Neurodegenerative Diseases

Some neurodegenerative diseases are caused by gain-of-function mutations. For example, SOD1G93A leads to development of amyotrophic lateral sclerosis (ALS). The system and method disclosed in this invention can be used to either correct the mutation or eliminate the mutant protein expression by introducing a stop codon or by changing a splicing site.

Cancers

Many genes (including tumor suppressor genes, oncogenes, and DNA repair genes) contribute to the development of cancer. Mutations in these genes often lead to various cancers. Using the system and method disclosed herein, one can specifically target and correct these mutations. As a result, causative oncogenic proteins can be functionally annulled or their expression can be eliminated by introducing a point mutation at either the catalytic sites or splicing sites. In some embodiments, the treatment, prophylaxis or diagnosis of cancer is provided. The target is preferably one or more of the FAS, BID, CTLA4, PDCD1, CBLB, PTPN6, TRAC, or TRBC genes. Cancer may be one or more of lymphoma, chronic lymphocytic leukemia (CLL), B cell acute lymphocytic leukemia (B-ALL), acute lymphoblastic leukemia, acute myeloid leukemia, non-Hodgkin's lymphoma (NHL), diffuse large cell lymphoma (DLCL), multiple myeloma, renal cell carcinoma (RCC), neuroblastoma, colorectal cancer, breast cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma. This may be implemented with engineered chimeric antigen receptor (CAR) T cell. This is described in WO2015161276, the disclosure of which is hereby incorporated by reference and described hereinbelow. Target genes suitable for the treatment or prophylaxis of cancer may include those described in WO2015048577, the disclosure of which is hereby incorporated by reference.

Stem Cell Genetic Modification

In some embodiments, stem cell or progenitor cell can be genetically modified using the system and method disclosed in this invention. Suitable cells include, e.g., stem cells (adult stem cells, embryonic stem cells, iPS cells, etc.) and progenitor cells (e.g., cardiac progenitor cells, neural progenitor cells, etc.). Suitable cells include mammalian stem cells and progenitor cells, including, e.g., rodent stem cells, rodent progenitor cells, human stem cells, human progenitor cells, etc. Suitable host cells include in vitro host cells, e.g., isolated host cells.

In some embodiments, the present invention can be used for targeted and precise genetic modification of tissue ex vivo, correcting the underlying genetic defects. After the ex vivo correction, the tissues may be returned to the patients. Moreover, the technology can be broadly used in cell-based therapies for correcting genetic diseases.

Genetic Editing in Animals and Plants

The system and method described above can be used to generate a transgenic non-human animal or plant having one or more genetic modification of interest. In some embodiments, the transgenic non-human animal is homozygous for the genetic modification. In some embodiments, the transgenic non-human animal is heterozygous for the genetic modification. In some embodiments, the transgenic non-human animal is a vertebrate, for example, a fish (e.g., zebrafish, goldfish, pufferfish, cavefish, etc.), an amphibian (frog, salamander, etc.), a bird (e.g., chicken, turkey, etc.), a reptile (e.g., snake, lizard, etc.), a mammal (e.g., an ungulate, e.g., a pig, a cow, a goat, a sheep, etc.; a lagomorph (e.g., a rabbit); a rodent (e.g., a rat, a mouse); or a non-human primate.

The invention can be used for treating diseases in animals in a way similar to those for treating diseases in humans, as described above. Alternatively, it can be used to generate knock-in animal disease models bearing specific genetic mutation(s) for purposes of research, drug discovery, and target validation. The system and method described above can also be used for introduction of point mutations to ES cells or embryos of various organisms, for the purpose of breeding and improving animal stocks and crop quality.

Methods of introducing exogenous nucleic acids into plant cells are well known in the art. Suitable methods include viral infection (such as double-stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e., in vitro, ex vivo, or in vivo).

### C. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

As used herein, the term“recombinant expression system” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell, such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively, the proteins produced may act as toxins under similar circumstances.

“Plasmids” used in genetic engineering are called “plasmid vectors.” Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

The term “single nucleotide polymorphism” or “SNP” refers to a change of a single nucleotide with a polynucleotide, including within an allele. This can include the replacement of one nucleotide by another, as well as deletion or insertion of a single nucleotide. Most typically, SNPs are biallelic markers, although tri- and tetra-allelic markers can also exist. By way of non-limiting example, a nucleic acid molecule comprising SNP A\C may include a C or A at the polymorphic position.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas polynucleotides are derived from the wild type Cas protein amino acid sequence. As used herein, the term “wild type” is a term of the art understood by skilled persons and means the typical form of an organism, strain, gene, or characteristic as it occurs in nature as distinguished from mutant or variant forms.

The term “isolated” when referring to nucleic acid molecules or polypeptides means that the nucleic acid molecule or the polypeptide is substantially free from at least one other component with which it is associated or found together in nature.

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to or modify using a CRISPR system. A target sequence may be within a nucleic acid in vitro or in vivo within the genome of a cell, which may be any form of single-stranded or double-stranded nucleic acid.

A “target nucleic acid strand” refers to a strand of a target nucleic acid that is subject to base-pairing with a crRNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and, thus, are still capable of nucleobase complementarity.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “treatment” or “treating,” or “palliating” or “ameliorating” are used interchangeably. These terms refer to an approach for obtaining beneficial or desired results, including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “prevent,” “preventing,” “prevention,” “prophylactic treatment” and the like refer to reducing the probability of developing a disorder or condition (e.g., SARS-CoV-2 infection) in a subject, who does not have, but is at risk of or susceptible to developing a disorder or condition. The term includes prevention of spread of infection in a subject exposed to the virus or at risk of having SARS-CoV-2 infection.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as a Cas protein or guide RNA (or crRNA), includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution or a number of different elements attached to a solid support at random or in no particular order in which the different elements are not spatially distinct. In other words, a mixture is not addressable.

The term “progeny,” such as the progeny of a transgenic plant, is one that is born of, begotten by, or derived from a plant or the transgenic plant. The introduced nucleic acid molecule may also be transiently introduced into the recipient cell such that the introduced nucleic acid molecule is not inherited by subsequent progeny and thus not considered “transgenic.” Accordingly, as used herein, a “non-transgenic” plant or plant cell is a plant that does not contain a foreign nucleic acid stably integrated into its genome.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “modulate” is meant to refer to any change in biological state, i.e., increasing, decreasing, and the like.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders, and/or who has been diagnosed with inflammatory disorders. In some embodiments, the subject is a mammal. In some embodiments, the subject is human.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in some embodiments,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of the invention.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 4%1, 3%1, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. EXAMPLES

### Example 1

This example describes the materials and methods used in the subsequent EXAMPLE(S) below.

Cas9 proteins used in the following experiments are Alt-R® S.p. HiFi Cas9 Nuclease V3 (catalog number 1081061 or 1081060 or 10007803 (IDT)) and Alt-R® S.p. dCas9 Protein V3 (catalog number 1081066 or 1081067 (IDT)). Ultramer donor oligos were synthesized and purified by IDT; Oligo R478X 5′-GTCCTCTCCGGGCTGGCCCAGATGATGAGAGAGTGCTGGTACCCCAACCCCTCTGCT tGaCTCACCGCACTGCGCATAAAGAAGACATTGCAGAAGCTCAGTCACAATCCAGAG AAGCCCAAAGTG-3′ (lower case nucleotides indicate the desired changes to make in the targeted allele) (SEQ ID NO: 1). The sgRNA (C473) was synthesized with this 20 nt Cas9 spacer sequence TCTTTATGCGCAGTGCGGTG (SEQ ID NO: 2) (MilliporeSigma) as a full-length sgRNA. HifiCas9 and dCas9 were mixed at indicated ratios with 6-fold molar excess sgRNA and electroporated or microinjected into C576BL/6J mouse embryos. Electroporated/microinjected embryos were transferred into psudoepregnant mouse recipients and embryos were carried to term. Live born mice were genotyped for the presence of mutated alleles by PCR using primers ACVRL1A 5′-CTGCTATGTCTCCCGATCCTGAG-3′ (SEQ ID NO: 3) and ACVRL1B 5′-CTCAGCTGTATTTTTGGCTGGATG-3′ (SEQ ID NO: 4) or ACVRL1C 5′-ATCACTTTGGGCTTCTCTGGATTG-3′ (SEQ ID NO: 5). Sequence analysis was done by NGS Amplicon Sequencing (Genewiz). All procedures involving animals were performed in accordance with Rutgers University IACUC policies on animal use.

### Example 2

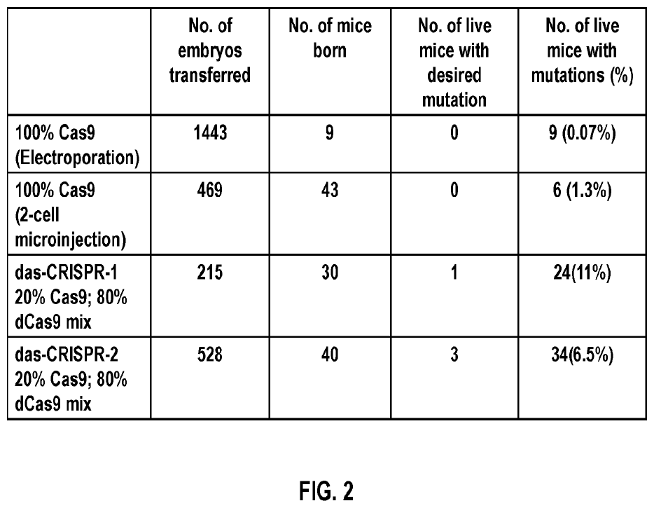

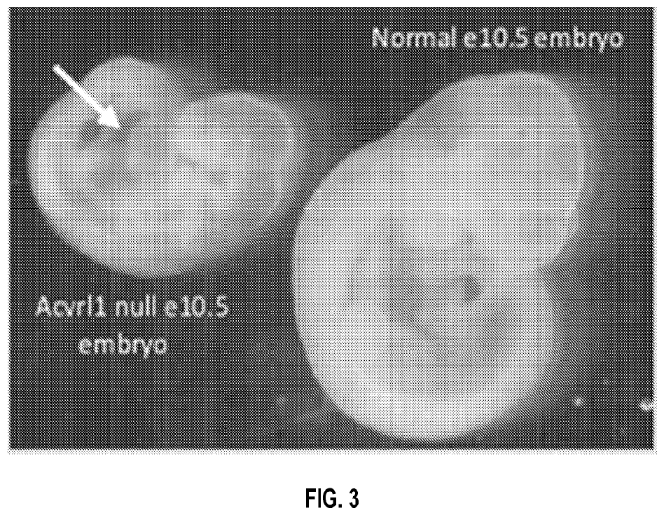

This Example demonstrates generating a mouse model using the disclosed system, as shown in FIG. 1. The results of a series of attempts to generate a mouse model of hereditary hemorrhagic telangiectasia by introducing a stop codon at amino acid position 478 are outlined in FIG. 2. Electroportaing or microinjecting the Cas9-sgRNA and donor oligo resulted in very few live births, indicating modifications were being made to the locus which caused embryonic lethality (example of malformed embryo in FIG. 3). Upon use of a Cas9:dCas9 ratio of 1:4, there was a significant increase in the number of live animals born with any type of mutation (example of malformed embryo in FIG. 3). Sequence alignments reveals successful gene editing of the Acvrl1 locus to create the R478X allele. The R478X allele is a stop codon which prematurely terminates translation of Acvrl1. NGS sequencing of the single founder from Experiment 3, dAS-CRISPR 1, showing the R478X mutation at a detectable frequency (SEQ ID NO: 14) and an intact wild-type allele (SEQ ID NO: 12), necessary for survival. NGS sequencing of three founders from Experiment 4, dAS-CRISPR 4, showing the R478X mutation at a detectable frequency in each founder and an intact wild-type allele (WT), which is necessary for survival. Other mutations (deletions, Δ; insertions, +; sequence changes, X>Y) are indicated. Upon further analysis, we found mice that harbored the R478X change at the Acvrl1 locus. Founder #172 from experiment dAS-CRISPR 1 was bred and transmitted the R478X allele. Founders from dAS-CRISPR 2 are also being tested for germline transmission.

The method outlined in this invention preserves one intact wildtype allele, which allows the cell or embryo to survive. This is done using dCas9, which is a catalytically inactivated Cas9 protein that retains its function to bind CRISPR gRNAs and to strongly bind to DNA, hence sequestering or blocking one allele from the action of an active nuclease Cas9. The inventors accomplish this by adjusting the ratio of functional Cas9:dCas9. When a mixture of 20% functional Cas9 and 80% dCas9 is used, the desired research model can be generated. Reagents are introduced into by electroporation into 1-cell embryos or injected into a single cell of a 2-cell embryo (to prevent modifying the entire embryo). Precise modifications were only obtained when dCAS9 was utilized in conjunction with functional Cas9.

dasCRISPR is a novel method to control the activity of Cas9 and allow successful creation of research models that may present a challenge with traditional CRISPR methods. Commercial enterprises that create mouse models would benefit from this method, allowing successful completion of projects. By protecting 1 of the 2 copies of a gene in vivo using dasCRISPR, a genetic change can be made that would otherwise be deleterious if both copies of the gene are modified. The same can be surmised for gene editing done in cell lines, which is a major service performed by biotech companies worldwide and in university facilities. Therapeutic gene editing would also benefit as this method could block access to some off-target sites by Cas protein and act as a brake in the gene editing process limiting modification to one allele while leaving the other off-target sites unaffected, when necessary. Some potential examples include: reduction of p53 activation by blocking sites, repairing only one allele; reduction of DNA damage response (DDR) by blocking cleavage at both alleles; or reduction of low specificity targeting in hematopoietic stem cells.

There are several applications for this method in production of research models and in therapeutic gene editing. 25-30% of gene knockouts in mouse, for example, lead to embryonic lethality, more when considering perinatal lethality and 7% result in infertility. These are barriers to propagation of research cell lines and animal models. By protecting 1 of the 2 copies of a gene in vivo using dasCRISPR, a genetic change can be made that would otherwise be deleterious if both copies of the gene are modified. The same can be surmised for gene editing done in cell lines, which is a major service performed by biotech companies worldwide and in university facilities.

TABLE 1
Representative nucleic acid and amino acid sequences

| | Founder Acvrl1 | | | | | |
|---|---|---|---|---|---|---|
| | Sequences | | | | | |
| | (Underlined | SEQ | | | | SEQ |
| Founder | is gRNA | ID | Muta- | | Amino Acid | ID |
| Alleles | sequence) | NO | tion | Frequency | Sequence | NO |
| WT | ACCCCAACCCCTCTG | 6 | N/A | N/A | VLSGLAQMM | 11 |
| Ref. | CTCGCCTCACCGCAC | | | | RECWYPNPSA | |
| | TGCGCATAAAGAAG | | | | RLTALRIKKTL | |
| | ACATTGCAGAAGCTC | | | | QKLSHNPEKP | |
| | AGTCACAATCCAGA | | | | K | |
| | GAAGCCCAAAG | | | | | |
| 172-1 | ACCCCAACCCCTCTG | 7 | WT | .57 | VLSGLAQMM | 12 |
| | CTCGCCTCACCGCAC | | | | RECWYPNPSA | |
| | TGCGCATAAAGAAG | | | | RLTALRIKKTL | |
| | ACATTGCAGAAGCTC | | | | QKLSHNPEKP | |
| | AGTCACAATCCAGA | | | | K | |
| | GAAGCCCAAAG | | | | | |
| 172-2 | ACCCCAACCCCTCTG | 8 | Δ1 | .18 | VLSGLAQMM | 13 |
| | CTCGCCTCA- | | | | RECWYPNPSA | |
| | CGCACTGCGCATAA | | | | RLTHCA*RRH | |
| | AGAAGACATTGCAG | | | | CRSSVTIQRSP | |
| | AAGCTCAGTCACAAT | | | | K | |
| | CCAGAGAAGCCCAA | | | | | |
| | AG | | | | | |
| 172-3 | ACCCCAACCCCTCTG | 9 | R478 | .17 | VLSGLAQMM | 14 |
| | CTTGACTCACCGCAC | | X | | RECWYPNPSA | |
| | TGCGCATAAAGAAG | | | | *LTALRIKKTL | |
| | ACATTGCAGAAGCTC | | | | QKLSHNPEKP | |
| | AGTCACAATCCAGA | | | | K | |
| | GAAGCCCAAAG | | | | | |
| 172-4 | ACCCCAACCCCTCTG | 10 | +1 | .07 | VLSGLAQMM | 15 |
| | CTCGCCTCACcCGCA | | | | RECWYPNPSA | |
| | CTGCGCATAAAGAA | | | | RLTRTAHKEDI | |
| | GACATTGCAGAAGC | | | | AEAQSQSREA | |
| | TCAGTCACAATCCAG | | | | Q | |
| | AGAAGCCCAAAG | | | | | |
| WT | ACCCCAACCCCTCTGCT | 6 | N/A | N/A | VLSGLAQM | 32 |
| Ref | CGCCTCACCGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 219-1 | ACCCCAACCCCTCTGCT | 16 | WT | .44 | VLSGLAQM | 33 |
| | CGCCTCACCGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 219-2 | ACCCCAACCCCTCTGCT | 17 | +7 | .22 | VLSGLAQM | 34 |
| | CGCCTCACTtctttatG | | | | MRECWYP | |
| | CACTGCGCATAAAGAAG | | | | NPSARLTSL | |
| | ACATTGCAGAAGCTCAG | | | | CTAHKEDI | |
| | TCACAATCCAGAGAAGC | | | | AEAQSQSR | |
| | CCAAAG | | | | EAQ | |
| 219-3 | ACCCCAACCCCTCTGCT | 18 | +15 | .15 | VLSGLAQM | 35 |
| | CGCCtcactcgacctcg | | | | MRECWYP | |
| | acTCACCGCACTGCGCA | | | | NPSARLTRP | |
| | TAAAGAAGACATTGCAG | | | | RLTALRIKK | |
| | AAGCTCAGTCACAATCC | | | | TLQKLSHN | |
| | AGAGAAGCCCAAAG | | | | PEKPK | |
| 219-4 | ACCCCAACCCCTCTGCT | 19 | R478X | .15 | VLSGLAQM | 36 |
| | TGACTCACCGCACTGCG | | | | MRECWYP | |
| | CATAAAGAAGACATTGC | | | | NPSA* | |
| | AGAAGCTCAGTCACAAT | | | | | |
| | CCAGAGAAGCCCAAAG | | | | | |
| 219-5 | ACCCCAACCCCTCTGCT | 20 | C > A | .04 | VLSGLAQM | 37 |
| | CGCCTCACAGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 223-1 | ACCCCAACCCCTCTGCT | 21 | WT | .90 | VLSGLAQM | 38 |
| | CGCCTCACCGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 223-2 | ACCCCAACCCCTCTGCT | 22 | Δ5 | .10 | VLSGLAQM | 39 |
| | CGCCT---- | | | | MRECWYP | |
| | CACTGCGCATAAAGAA | | | | NPSARLTA | |
| | GACATTGCAGAAGCTCA | | | | HKEDIAEA | |
| | GTCACAATCCAGAGAA | | | | QSQSREAQ | |
| | GCCCAAAG | | | | | |
| 224-1 | ACCCCAACCCCTCTGCT | 23 | WT | .94 | VLSGLAQM | 40 |
| | CGCCTCACCGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 224-2 | ACCCCAACCCCTCTGCT | 24 | Δ5 | .06 | VLSGLAQM | 41 |
| | CGCCT----- | | | | MRECWYP | |
| | CACTGCGCATAAAGAA | | | | NPSARLTA | |
| | GACATTGCAGAAGCTCA | | | | HKEDIAEA | |
| | GTCACAATCCAGAGAA | | | | QSQSREAQ | |
| | GCCCAAAG | | | | | |
| 239-1 | ACCCCAACCCCTCTGCT | 25 | WT | .59 | VLSGLAQM | 42 |
| | CGCCTCACCGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 239-2 | ACCCCAACCCCTCTGCT | 26 | +1 | .22 | VLSGLAQM | 43 |
| | CGCCTCACcCGCACTGC | | | | MRECWYP | |
| | GCATAAAGAAGACATT | | | | NPSARLTR | |
| | GCAGAAGCTCAGTCACA | | | | TAHKEDIA | |
| | ATCCAGAGAAGCCCAA | | | | EAQSQSRE | |
| | AG | | | | AQ | |
| 239-3 | ACCCCAACCCCTCTGCT | 27 | R478X | .18 | VLSGLAQM | 44 |
| | TGACTCACCGCACTGCG | | | | MRECWYP | |
| | CATAAAGAAGACATTGC | | | | NPSA* | |
| | AGAAGCTCAGTCACAAT | | | | | |
| | CCAGAGAAGCCCAAAG | | | | | |
| 239-4 | ACCCCAACCCCTCTGCT | 28 | Δ5 | .01 | VLSGLAQM | 45 |
| | CGCCT----- | | | | MRECWYP | |
| | CACTGCGCATAAAGAA | | | | NPSARLTA | |
| | GACATTGCAGAAGCTCA | | | | HKEDIAEA | |
| | GTCACAATCCAGAGAA | | | | QSQSREAQ | |
| | GCCCAAAG | | | | | |
| 246-1 | ACCCCAACCCCTCTGCT | 29 | WT | .74 | VLSGLAQM | 46 |
| | CGCCTCACCGCACTGCG | | | | MRECWYP | |
| | CATAAAGAAGACATTGC | | | | NPSARLTA | |
| | AGAAGCTCAGTCACAAT | | | | LRIKKTLQ | |
| | CCAGAGAAGCCCAAAG | | | | KLSHNPEK | |
| | | | | | PK | |
| 246-2 | ACCCCAACCCCTCTGCT | 30 | R478X | .17 | VLSGLAQM | 47 |
| | TGACTCACCGCACTGCG | | | | MRECWYP | |
| | CATAAAGAAGACATTGC | | | | NPSA* | |
| | AGAAGCTCAGTCACAAT | | | | | |
| | CCAGAGAAGCCCAAAG | | | | | |
| 246-3 | ACCCCAACCCCTCTGCT | 31 | +1 | .09 | VLSGLAQM | 48 |
| | CGCCTCACcCGCACTGC | | | | MRECWYP | |
| | GCATAAAGAAGACATT | | | | NPSARLTR | |
| | GCAGAAGCTCAGTCACA | | | | TAHKEDIA | |
| | ATCCAGAGAAGCCCAA | | | | EAQSQSRE | |
| | AG | | | | AQ | |

1. A system for gene editing, comprising:
a CRISPR-associated protein (Cas) polypeptide or a first Cas nucleotide sequence encoding a Cas polypeptide;
a nuclease-deficient Cas (dCas) polypeptide or a second Cas nucleotide sequence encoding a dCas polypeptide; and
a guide nucleotide sequence encoding or comprising a crRNA sequence capable of hybridizing with a first target sequence on a first allele and a second target sequence on a second allele and forming a complex with the Cas polypeptide and the dCas polypeptide,
wherein the Cas polypeptide binds to the first target sequence on the first allele and induces genetic modification in the first target sequence, and
wherein the dCas polypeptide binds to the second target sequence on the second allele and protects the second target sequence from modification and from the activity of the Cas polypeptide

2. The system of claim 1, wherein the first target sequence comprises one or more mutations.

3. The system of claim 1, wherein the first target sequence and the second target sequence are identical or the first target sequence comprises one or more mutations with respect to the second target sequence.

4. The system of claim 1, wherein the genetic modification comprises an insertion of a stop codon, a point mutation, a deletion or an insertion.

5. The system of claim 1, wherein the guide nucleotide sequence together with the Cas polypeptide or the dCas polypeptide are delivered to a cell or an embryo as a ribonucleoprotein complex.

6. The system of claim 1, wherein the Cas polypeptide and the dCas polypeptide have a ratio of between about 1:100 and about 100:1.

7. The system of claim 6, wherein the Cas polypeptide and the dCas polypeptide have a ratio of between about 1:10 and about 10:1, and optionally wherein the Cas polypeptide and the dCas polypeptide have a ratio of about 2:1, about 1:2, about 1:4, about 1:6, or about 1:8.

8. The system of claim 1, wherein the first Cas nucleotide sequence and the second Cas nucleotide sequence are located on the same vector.

9. The system of claim 1, wherein the guide nucleotide sequence is located on the same vector with the first Cas nucleotide sequence or with the second Cas nucleotide.

10. The system of claim 9, further comprising a second guide nucleotide sequence, wherein the second guide nucleotide sequence and the second Cas nucleotide sequence are located on the same vector, and wherein the guide nucleotide sequence and the first Cas nucleotide sequence are located on the same vector.

11. The system of claim 1, wherein the guide nucleotide sequence is a synthetic RNA molecule.

12. The system of claim 1, wherein the first Cas nucleotide or the Cas polypeptide sequence and the second Cas nucleotide or the dCas polypeptide sequence are otherwise identical, except that the second Cas nucleotide or the dCas polypeptide comprises one or more mutations causing a deficiency in nuclease activity of the dCas polypeptide.

13. The system of claim 1, wherein the Cas polypeptide and the dCas polypeptide belong to different CRISPR CAS families.

14. The system of claim 1, wherein the Cas polypeptide is selected from the group consisting of a Cas9 nuclease, a Cpf1 nuclease, a Cas12a nuclease, a Cas12e nuclease, a CasX nuclease, a Cas12d nuclease, a CasY nuclease, a Cas12b nuclease, a C2C1 nuclease, a Cas12c nuclease, a C2C3 nuclease, a C2C4 nuclease, a C2C5 nuclease, a C2C6 nuclease, a C2C7 nuclease, a C2C8 nuclease, a C2C9 nuclease, a C2C10 nuclease, a Cas13a nuclease, a Cas13b nuclease, and a Cas13c nuclease.

15. The system of claim 1, wherein the Cas polypeptide is a Cas9 nuclease.

16. The system of claim 14, wherein the Cas9 nuclease is selected from the group consisting of Streptococcus pyogenes Cas9 (SpCas9), Staphylococcus aureus Cas9 (SaCas9), Neisseria meningitidis Cas9 (NmCas9), Actinomyces naeslundii Cas9 (AnCas9), and Streptococcus thermophilus Cas9 (StCas9).

17. A host cell or cell line or progeny thereof comprising the system of claim 1.

18. The host cell or cell line or progeny thereof of claim 17, comprising a stem cell or stem cell line.

19. A composition comprising the system of claim 1.

20. A method of modifying a target sequence of interest comprising delivering the system of claim 1 to the target sequence or a cell containing the target sequence and thereby inducing a modification in the target sequence.

21. The method of claim 20, wherein the target sequence is located at genomic loci of interest.

22. The method of claim 20, wherein the target sequence is part of a gene and the modification in the target sequence modulates the expression level or the function of the gene.

23. The method of claim 22, wherein the modification in the target sequence increases or reduces the expression level or the function of the gene.

24. The method of claim 23, wherein the gene is selected from the group consisting of p53, LOXL1, NOX4, SNX27, and Cathepsin B.

25. The method of claim 24, wherein the modification on only one allele, while protecting the second allele, causes reduced DNA damage response (DDR) in hematopoietic stem cells and other cells, reduced cytokine expression, or reduced inflammation.

26. The method of claim 20, the cell is a eukaryotic cell.

27. The method of claim 26, wherein the cell is a plant, animal, or human cell.

28. The method of claim 20, comprising delivering the system via particles, vesicles, or one or more viral vectors.

29. The method of claim 28, wherein the one or more viral vectors comprise an adenovirus-based vector, a lentivirus-based vector, or an adeno-associated virus-based vector.

30. The method of claim 20, wherein the target sequence comprises a genetic defect that is associated with a disease or a naturally occurring variant not associated with a disease.

31. The method of claim 30, wherein the disease is cancer, a genetic disease or a neurodegenarative disease.

32. A method of treating a disease of a subject caused by a genetic defect in a target sequence, comprising: administering the system of claim 1 containing the target sequence in a subject in need thereof and thereby inducing a modification in the target sequence.

33. The method of claim 32, wherein the target sequence is located at genomic loci of interest.

34. The method of claim 32, wherein the target sequence is part of a gene and the modification in the target sequence modulates the expression level and the function of the gene.

35. The method of claim 34, wherein the gene is selected from the group consisting of p53, LOXL1, NOX4, SNX27, and Cathepsin B.

36. The method of claim 34, wherein the modification causes reduced DNA damage response (DDR), reduced specificity in targeting in hematopoietic stem cells, reduced cytokine expression, or reduced inflammation.

37. The method of claim 32, the cell is a eukaryotic cell.

38. The method of claim 37, wherein the cell is a plant, animal, or human cell.

39. The method of claim 32, comprising delivering the system via particles, vesicles, or one or more viral vectors.

40. The method of claim 39, wherein the one or more viral vectors comprise an adenovirus-based vector, a lentivirus-based vector, or an adeno-associated virus-based vector.

41. The method of claim 32, wherein the disease is cancer, a genetic disease or a neurodegenerative disease.

42. A method for blocking a nucleotide sequence from cleavage, comprising: contacting a nucleic acid molecule comprising a target sequence that is subject to protection from cleavage by an endonuclease with (i) a dCas polypeptide and (ii) a guide nucleotide sequence encoding or comprising a crRNA sequence capable of hybridizing with a target sequence, wherein a complex of the dCas polypeptide and the guide nucleotide sequence binds to the target sequence, thereby blocking the target sequence from being cleaved by the endonuclease.


SEQUENCE LISTING (57 sequences)

SEQ ID NO: 1 - DNA, 126 bp, Artificial
  Synthetic
gtcctctccgggctggcccagatgatgagagagtgctggtaccccaacccctctgcttgactcaccgcactgcgcataaagaagacattgcagaagctcagtcacaatccagagaagcccaaagtg

SEQ ID NO: 2 - DNA, 20 bp, Artificial
  Synthetic
tctttatgcgcagtgcggtg

SEQ ID NO: 3 - DNA, 23 bp, Artificial
  Synthetic
ctgctatgtctcccgatcctgag

SEQ ID NO: 4 - DNA, 24 bp, Artificial
  Synthetic
ctcagctgtatttttggctggatg

SEQ ID NO: 5 - DNA, 24 bp, Artificial
  Synthetic
atcactttgggcttctctggattg

SEQ ID NO: 6 - DNA, 84 bp, Artificial
  Synthetic
accccaacccctctgctcgcctcaccgcactgcgcataaagaagacattgcagaagctcagtcacaatccagagaagcccaaag

SEQ ID NO: 7 - DNA, 84 bp, Artificial; identical to SEQ ID NO: 6
  Synthetic

SEQ ID NO: 8 - DNA, 83 bp, Artificial
  Synthetic
accccaacccctctgctcgcctcacgcactgcgcataaagaagacattgcagaagctcagtcacaatccagagaagcccaaag

SEQ ID NO: 9 - DNA, 84 bp, Artificial
  Synthetic
accccaacccctctgcttgactcaccgcactgcgcataaagaagacattgcagaagctcagtcacaatccagagaagcccaaag

SEQ ID NO: 10 - DNA, 85 bp, Artificial
  Synthetic
accccaacccctctgctcgcctcacccgcactgcgcataaagaagacattgcagaagctcagtcacaatccagagaagcccaaag

SEQ ID NO: 11 - protein, 41 aa, Artificial
  Synthetic
VLSGLAQMMRECWYPNPSARLTALRIKKTLQKLSHNPEKPK

SEQ ID NO: 12 - protein, 41 aa, Artificial; identical to SEQ ID NO: 11
  Synthetic

SEQ ID NO: 13 - protein, 25 aa, Artificial
  Synthetic
VLSGLAQMMRECWYPNPSARLTHCA

SEQ ID NO: 14 - protein, 19 aa, Artificial
  Synthetic
VLSGLAQMMRECWYPNPSA

SEQ ID NO: 15 - protein, 41 aa, Artificial
  Synthetic
VLSGLAQMMRECWYPNPSARLTRTAHKEDIAEAQSQSREAQ

SEQ ID NO: 16 - DNA, 84 bp, Artificial; identical to SEQ ID NO: 6
  Synthetic

SEQ ID NO: 17 - DNA, 91 bp, Artificial
  Synthetic
accccaacccctctgctcgcctcacttctttatgcactgcgcataaagaagacattgcagaagctcagtcacaatccagagaagcccaaag

SEQ ID NO: 18 - DNA, 99 bp, Artificial
  Synthetic
accccaacccctctgctcgcctcactcgacctcgactcaccgcactgcgcataaagaagacattgcagaagctcagtcacaatccagagaagcccaaag

SEQ ID NO: 19 - DNA, 84 bp, Artificial; identical to SEQ ID NO: 9
  Synthetic

SEQ ID NO: 20 - DNA, 84 bp, Artificial
  Synthetic
accccaacccctctgctcgcctcacagcactgcgcataaagaagacattgcagaagctcagtcacaatccagagaagcccaaag

SEQ ID NO: 21 - DNA, 84 bp, Artificial; identical to SEQ ID NO: 6
  Synthetic

SEQ ID NO: 22 - DNA, 79 bp, Artificial
  Synthetic
accccaacccctctgctcgcctcactgcgcataaagaagacattgcagaagctcagtcacaatccagagaagcccaaag

SEQ ID NO: 23 - DNA, 84 bp, Artificial; identical to SEQ ID NO: 6
  Synthetic

SEQ ID NO: 24 - DNA, 79 bp, Artificial; identical to SEQ ID NO: 22
  Synthetic

SEQ ID NO: 25 - DNA, 84 bp, Artificial; identical to SEQ ID NO: 6
  Synthetic

SEQ ID NO: 26 - DNA, 85 bp, Artificial; identical to SEQ ID NO: 10
  Synthetic

SEQ ID NO: 27 - DNA, 84 bp, Artificial; identical to SEQ ID NO: 9
  Synthetic

SEQ ID NO: 28 - DNA, 79 bp, Artificial; identical to SEQ ID NO: 22
  Synthetic

SEQ ID NO: 29 - DNA, 84 bp, Artificial; identical to SEQ ID NO: 6
  Synthetic

SEQ ID NO: 30 - DNA, 84 bp, Artificial; identical to SEQ ID NO: 9
  Synthetic

SEQ ID NO: 31 - DNA, 85 bp, Artificial; identical to SEQ ID NO: 10
  Synthetic

SEQ ID NO: 32 - protein, 41 aa, Artificial; identical to SEQ ID NO: 11
  Synthetic

SEQ ID NO: 33 - protein, 43 aa, Artificial
  Synthetic
VLSGLAQMMRECWYPNPSARLTSLCTAHKEDIAEAQSQSREAQ

SEQ ID NO: 34 - protein, 43 aa, Artificial; identical to SEQ ID NO: 33
  Synthetic

SEQ ID NO: 35 - protein, 46 aa, Artificial
  Synthetic
VLSGLAQMMRECWYPNPSARLTRPRLTALRIKKTLQKLSHNPEKPK

SEQ ID NO: 36 - protein, 19 aa, Artificial
  Synthetic
VLSGLAQMMRECWYPNPSA

SEQ ID NO: 37 - protein, 41 aa, Artificial; identical to SEQ ID NO: 11
  Synthetic

SEQ ID NO: 38 - protein, 41 aa, Artificial; identical to SEQ ID NO: 11
  Synthetic

SEQ ID NO: 39 - protein, 39 aa, Artificial
  Synthetic
VLSGLAQMMRECWYPNPSARLTAHKEDIAEAQSQSREAQ

SEQ ID NO: 40 - protein, 41 aa, Artificial; identical to SEQ ID NO: 11
  Synthetic

SEQ ID NO: 41 - protein, 39 aa, Artificial; identical to SEQ ID NO: 39
  Synthetic

SEQ ID NO: 42 - protein, 41 aa, Artificial; identical to SEQ ID NO: 11
  Synthetic

SEQ ID NO: 43 - protein, 41 aa, Artificial; identical to SEQ ID NO: 15
  Synthetic

SEQ ID NO: 44 - protein, 19 aa, Artificial
  Synthetic
VLSGLAQMMRECWYPNPSA

SEQ ID NO: 45 - protein, 39 aa, Artificial; identical to SEQ ID NO: 39
  Synthetic

SEQ ID NO: 46 - protein, 41 aa, Artificial; identical to SEQ ID NO: 11
  Synthetic

SEQ ID NO: 47 - protein, 19 aa, Artificial
  Synthetic
VLSGLAQMMRECWYPNPSA

SEQ ID NO: 48 - protein, 41 aa, Artificial; identical to SEQ ID NO: 15
  Synthetic

SEQ ID NO: 49 - protein, 4 aa, Artificial
  Synthetic
SGGS

SEQ ID NO: 50 - protein, 4 aa, Artificial
  Synthetic
SGGG

SEQ ID NO: 51 - protein, 5 aa, Artificial
  Synthetic
SGGGS

SEQ ID NO: 52 - protein, 5 aa, Artificial
  Synthetic
SGGGG

SEQ ID NO: 53 - protein, 6 aa, Artificial
  Synthetic
SGGGGS

SEQ ID NO: 54 - protein, 6 aa, Artificial
  Synthetic
SGGGGG

SEQ ID NO: 55 - protein, 7 aa, Artificial
  Synthetic
SGGGGGS

SEQ ID NO: 56 - protein, 7 aa, Artificial
  Synthetic
SGGGGGG

SEQ ID NO: 57 - protein, 8 aa, Artificial
  Synthetic
SGGGGGGS